(19) United States
(12) Patent Application Publication
(10) Pub. No.: US 2007/0275426 A1
(43) Pub. Date: Nov. 29, 2007

(54) Disk-like microfluidic structure for generating diffrent concentration fluid mixtures

(76) Inventors: Andrew M. Wo, Taipei City (TW); Chen-Lin Chen, Taipei City (TW); Yi-Fang Chang, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/882,965

(22) Filed: Aug. 8, 2007

(30) Foreign Application Priority Data
Apr. 3, 2006 (TW) 96111863

(51) Int. Cl. G01N 33/00

(52) U.S. Cl. 435/7.92

(57) ABSTRACT

A disk-like microfluidic structure includes a disk, and a plurality of microfluidic systems formed on the disk and covered by a top sealing layer. Each microfluidic system includes a first and a second supply chamber located near a geometrical center of the disk, and at least one receiving chamber communicably connected to the first and the second supply chambers via a first and a second microchannel, respectively. When the first and the second microchannels have different geometrical sizes and the disk is driven to spin, two fluids separately held in the first and the second supply chambers are centrifugally moved into the receiving chamber via the differently sized first and second microchannels at different speeds and in different quantities to generate in the receiving chamber a fluid mixture having a specific concentration. By changing the geometrical sizes of the first and second microchannels, different concentration fluid mixtures may be generated.

### FIELD OF THE INVENTION

The present invention relates to a microfluidic platform that can be spun to generate fluid mixtures having different specific concentrations, and more particularly, to a disk-like microfluidic structure for generating different concentration fluid mixtures.

### BACKGROUND OF THE INVENTION

In different chemical, medical, and biological experiments, it is often necessary to prepare a plurality of specimens or samples of specific concentrations for conducting related concentration analyses or experiments. Conventionally, the specimens of different specific concentrations are manually prepared using droppers or pipettes. Therefore, a lot of labor and time are required.

With the development in micro-electromechanical techniques, various kinds of microfluidic chips have been introduced into the market, such as concentration gradient microfluidic chips based on a polydimethylsiloxane (PDMS) platform. Fluid samples are injected and pushed to the platform using a syringe pump. With multiple layers of microfluidic branches and zigzag microchannels formed on the microfluidic chip, the fluid samples flowing through the microchannels are diluted and mixed, and samples of different concentrations may be obtain at the downstream of the microchannels. To proceed the analyses of samples, the various fluid samples are treated with reagents to react and the results are analyzed. The chip is designed to be an integral structure having microchannels and micro-reactors, so that the fluid samples are transported through the array platform and mixed by centrifugal force and Coriolis force. The various samples are then treated with reagents to react and the results are analyzed. The whole assays proceed in short time with reduced use of samples and reagents. Moreover, a large quantity of parallelized experimental results may be obtained.

U.S. Pat. No. 5,061,381 discloses a technical means for separating plasma from whole blood using centrifugal force by a centrifugal rotor which includes a measuring chamber, an overflow chamber, a separation chamber, and a reagent chamber. The measuring chamber and the overflow chamber have a plurality of microchannels. The separation chamber includes a plurality of radially arranged passages and is used to separate plasma from the whole blood, so as to perform blood assays.

U.S. Pat. No. 5,242,803 discloses a disk-shaped fluid array platform. Blood sample is positioned in a central chamber, to which a plurality of other chambers are radially connected. When the platform is spun, the blood sample is driven to flow by centrifugal force from the central chamber to the other chambers where the biochemical analyses and experiments are carried out.

U.S. Pat. No. 6,399,361 discloses devices and methods for using centripetal acceleration to drive fluid movement in a microfluidic system. The microfluidic system includes microfluidic components, such as temperature sensing elements, mixing structures, valves, etc., and is used for performing biochemical and chemical experiments. The microfluidic system may be an antibiotic assay platform, a blood separation array platform, a mixing array platform, an immunoassay platform, a thermal valve, etc.

Taiwan Invention Publication Nos. 200528718 and 200528727 disclose a method using centrifugal type microchannel chip and a portable apparatus possessing the microchannel chip. In the invention, a liquid sample is driven by centrifugal force at high speed spinning to pass through microchannels. With different passages, channels, and capillary switches, testing samples react step by step to perform the testing.

However, it is uneasy for the conventional carrier platforms to complete the pumping and mixing of microfluids. Therefore, it is necessary to integrate a micropump or micromixer onto the chip. However, with this design, the micropump must continuously deliver the samples into the microchannels, and therefore, a large quantity of sample is required and consumed. In addition, it is uneasy to achieve uniform mixing of samples in the microchannels.

Moreover, when using pipette to measure and transfer a liquid sample, the sample has to be put into a container for carrying out the subsequent experiments. It requires a lot of preparatory works and accordingly, a lot of time and procedures to manually handle the dropper or pipette. And, cares must be taken to ensure the dropper or the pipette to be used in each preparation has been thoroughly cleaned without residual sample and to prevent operating errors, so as to avoid adverse influences on the accuracy of the analysis.

It is therefore desired to develop a microfluidic platform that can generate different concentration fluid mixtures simultaneously and rapidly for proceeding analyses.

### SUMMARY OF THE INVENTION

A primary object of the present invention is to provide a disk-like microfluidic platform with driving device. The microfluidic platform is provided with microchannels, supply chambers, and receiving chambers. When the disk-like microfluidic platform is driven by the driving device to spin, fluids supplied to the microfluidic platform are driven to flow centrifugally from the supply chambers through the microchannels into the receiving chambers, and well mixed under the action of Coriolis force.

Another object of the present invention is to provide a disk-like microchannel structure, which includes a microfluidic platform provided with straight or bent microchannels having different geometrical lengths, widths, and depths, and supply and receiving chambers having different geometrical shapes and locations, as well as straight or bent mixing channels or convergent points of many branch microchannels, so that different concentration fluid mixtures can be generated using the disk-like microfluidic structure.

In a disk-like microfluidic structure according to a preferred embodiment of the present invention, a plurality of microfluidic systems are arranged on a microchannel forming surface of a disk as a radial array to equally space from one another by a predetermined angle. Each of the microfluidic systems includes at least a first and a second supply chamber for holding a first and a second fluid, respectively; a first receiving chamber communicably connected to the first and the second supply chamber via a first and a second microchannel, respectively; and a second receiving chamber communicably connected to the first and the second supply chambers via a third and a fourth microchannel, respectively. The microchannels may be straight or bent, and the supply and the receiving chambers may have different geometrical shapes and locations. By giving the first, the second, the third, and the fourth microchannel different geometrical sizes and structures, different concentration fluid mixtures of the first and the second fluids may be obtained at the first and the second receiving chambers.

In a disk-like microfluidic structure according to another embodiment of the present invention, the first and the second microchannels are converged at a first mixing channel before moving into the first receiving chamber; similarly, the third and the fourth microchannels are converged at a second mixing channel before moving into the second receiving chamber. The first and second mixing channels have the function of enhancing the mixing of the first fluid with the second fluid.

To fulfill the above objects, the present invention provides a disc-like microfluidic structure which comprises a disk, and a plurality of microfluidic systems formed on the disk and covered by a top sealing layer. Each microfluidic system includes a first and a second supply chamber located near a geometrical center of the disk, and at least one receiving chamber communicably connected to the first and the second supply chambers via a first and a second microchannel, respectively. When the first and the second microchannels have different geometrical sizes and the disk is driven to spin, two fluids separately held in the first and the second supply chamber centrifugally flow into the receiving chamber via the differently sized first and second microchannels at different speeds to the receiving chamber, generating a fluid mixture having a specific concentration. By changing the geometrical sizes of the first and second microchannels, different concentration fluid mixtures may be generated.

The disk-like microchannel structure for generating different concentration fluid mixtures according to the present invention not only enables improved efficiency in generating fluid specimens of different concentrations simultaneouly, but also easy integration with subsequent testing works, allowing the whole experiment to be conducted in a more convenient manner at reduced labors and time and with reduced use of fluid specimen and preventing man-made errors to meet the requirements of high efficiency, low cost, accuracy, and time saving. The present invention may be applied in chemical or biochemical experiments in which a plurality of different concentration solutions or specimens are to be generated for conducting related concentration analyses or experiments, such as the obtaining of a calibration curve, the cell culture, the optical inspection, etc. By changing the geometrical shapes or the arrangement of the microchannels, the supply chambers, and the receiving chambers in the microchannel platform of the present invention, a plurality of different concentration specimens may be obtained.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein:

FIG. 1 is an exploded perspective view of a disk-like microfluidic structure for generating different concentration fluid mixtures according to a first embodiment of the present invention;

FIG. 2 is a top view of a disk of the disk-like microfluidic structure in the first embodiment of the present invention;

FIG. 3 is a top view of a top sealing layer of the disk-like microfluidic structure in the first embodiment of the present invention;

FIG. 4 is a cross sectional view taken along line 4-4 of FIG. 2;

FIG. 5 is a cross sectional view taken along line 5-5 of FIG. 2;

FIG. 6 is a cross sectional view taken along line 6-6 of FIG. 2;

FIG. 7 is an exploded perspective view of a disk-like microfluidic structure for generating different concentration fluid mixtures according to a second embodiment of the present invention;

FIG. 8 is a top view of a disk of the disk-like microfluidic structure in the second embodiment of the present invention;

FIG. 9 is a top view of a top sealing layer of the disk-like microfluidic structure in the second embodiment of the present invention;

FIG. 10 is a cross sectional view taken along line 10-10 of FIG. 8;

FIG. 11 is a cross sectional view taken along line 11-11 of FIG. 8;

FIG. 12 is a top view of a disk with microfluidic systems included in a disk-like microfluidic structure for generating different concentration according to a third embodiment of the present invention; and

FIG. 13 is a cross sectional view taken along line 13-13 of FIG. 12 showing differently sized microchannels provided on the disk.

### DETAILED DESCRIPTION OF THE PREFERRED EMBODIMENTS

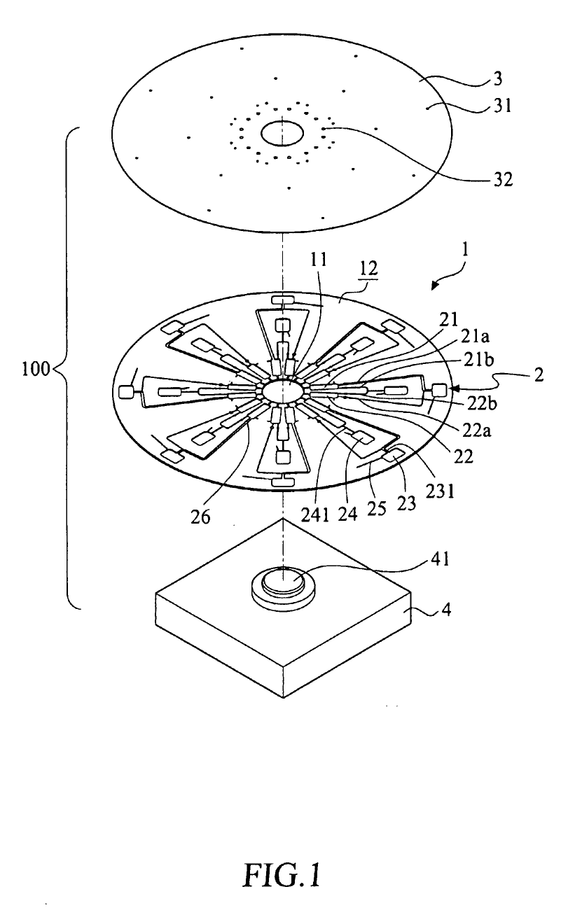

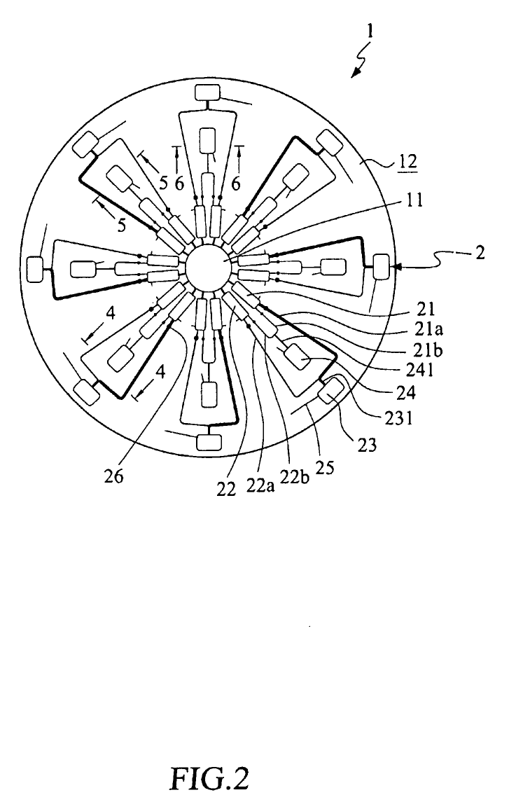

Please refer to FIG. 1 that is an exploded perspective view of a disk-like microfluidic structure 100 for generating different concentration fluid mixtures according to a first embodiment of the present invention; and to FIG. 2 that is a top view of a disk 1 included in the first embodiment of the present invention. As shown, the disk-like microfluidic structure 100 includes a disk 1, on which a plurality of microfluidic systems 2 are provided; a top sealing layer 3 covering one side of the disk 1; and a driving device 4 for driving the disk 1 to spin and thereby produce a centrifugal force. When the disk 1 spins, different concentration fluid mixtures may be generated with the microfluidic structure 100.

The disk 1 includes a central opening 11 and a microchannel forming surface 12, on which the plurality of microfluidic systems 2 are formed as a radial array to space from one another by a predetermined angle. Each of the microfluidic systems 2 includes a first supply chamber 21, a second supply chamber 22, a first microchannel 21a, a second microchannel 22a, a third microchannel 21b, a fourth microchannel 22b, a first mixing channel 231 at where the first and the second microchannels 21a, 22a are converged, a second mixing channel 241 at where the third and the fourth microchannels 21b, 22b are converged, a first receiving chamber 23, a second receiving chamber 24, and a plurality of exhaust channels 25.

The first supply chamber 21 is located near a geometric center of the disk 1, and is communicably connected to the first and the third microchannels 21a, 21b. The second supply chamber 22 is also located near the geometric center of the disk 1 to space from the first supply chamber 21 by a predetermined distance, and is communicably connected to the second and the fourth microchannels 22a, 22b. The first and the second microchannels 21a, 22a are converged into the first mixing channel 231, which is communicably connected to the first receiving chamber 23. On the other hand, the third and the fourth microchannels 21b, 22b are converged into the second mixing channel 241, which is communicably connected to the second receiving chamber 24.

The plurality of exhaust channels 25 are separately communicably connected to the first supply chamber 21, the second supply chamber 22, the first receiving chamber 23, and the second receiving chamber 24 for discharging gas produced in the microfluidic system 2 in the course of generating different concentration fluid mixtures.

When the disk 1 is driven by the driving device 4 to spin, fluids in the first and the second supply chamber 21, 22 are driven to flow by a centrifugal force through the first and second microchannels 21a, 22a into the first receiving chamber 23 to generate a fluid mixture of a first specific concentration, and through the third and fourth microchannels 21b, 22b into the second receiving chamber 24 to generate a fluid mixture of a second specific concentration. Further, the fluids driven to flow centrifugally from the first and the second supply chambers 21, 22 through the first and second microchannels 21a, 22a are converged at the first mixing channel 231 to well mix with each other under the Coriolis effect before flowing into the first receiving chamber 23, and the fluids driven to flow centrifugally from the first and the second supply chambers 21, 22 through the third and fourth microchannels 21b, 22b are converged at the second mixing channel 241 to well mix with each other under the Coriolis Effect before being moved into the second receiving chamber 24. Wherein, the first, the second, the third, and the fourth microchannels 21a, 22a, 21b, 22b may be straight or bent microchannels. And, the mixing channels 231, 241 may be straight or bent microchannels, or convergent points of a plurality of branch microchannels.

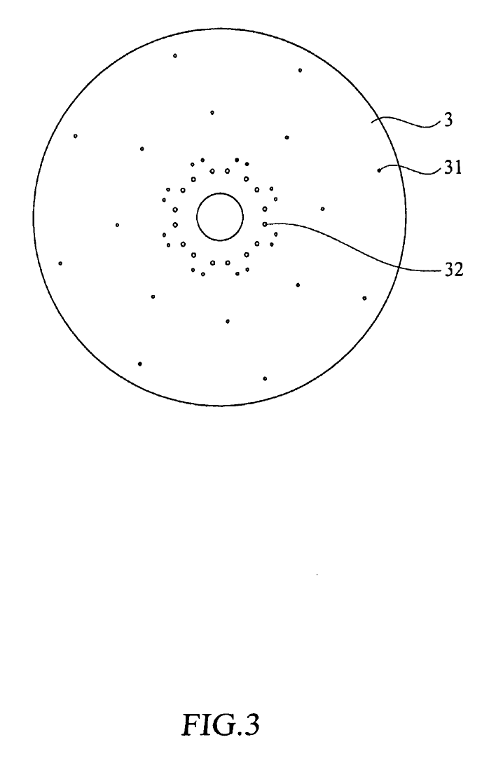

Please refer to FIG. 3 that is a top view of the top sealing layer 3 included in the first embodiment of the present invention for covering over the microchannel forming surface 12 of the disk 1, so as to prevent the fluids from flowing out of the microfluidic systems 2. On the top sealing layer 3, there are provided a plurality of venting pores 31 and a plurality of inlet ports 32. The venting pores 31 are located corresponding to the exhaust channels 25 of the microfluidic systems 2.

The top sealing layer 3 may be attached to the disk 1 by thermal bonding, bonding through surface modification, or adhesive bonding.

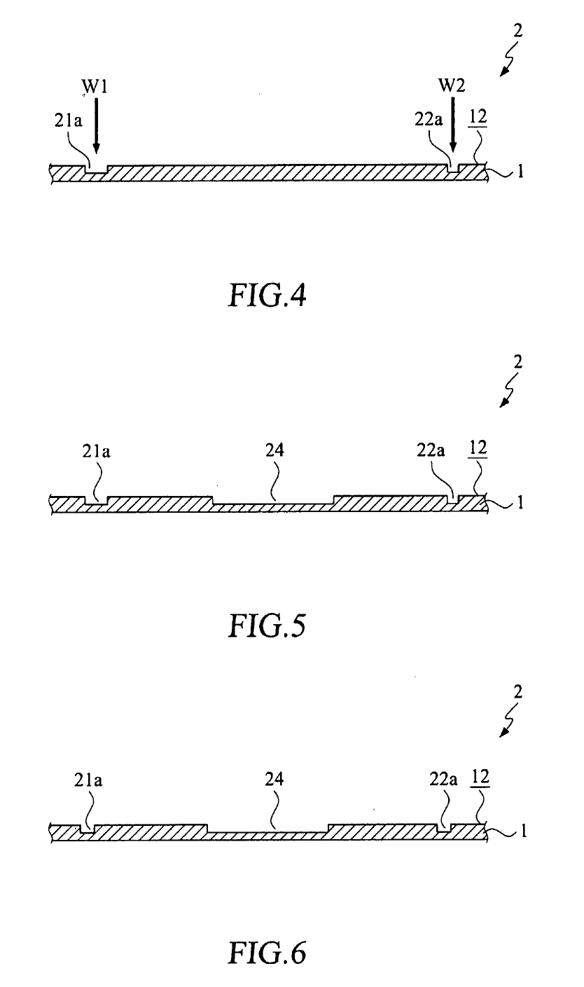

FIG. 4 is a cross sectional view taken along line 4-4 of FIG. 2. The first and the second supply chambers 21, 22 in each of the microfluidic systems 2 have a first fluid W1 and a second fluid W2 held therein, respectively. The inlet ports 32 on the top sealing layer 3 are located corresponding to the first and the second chambers 21, 22 formed on the disk 1, and the fluids W1, W2 are respectively supplied into the first and the second supply chambers 21, 22 via the inlet ports 32.

The driving device 4 is mounted to one surface of the disk 1 opposite to the microchannel forming surface 12, and includes a driving shaft 41 corresponding to the central opening 11 of the disk 1. The driving device 4 may be a motor or a CD driver. After the first and the second fluids W1, W2 are supplied to the first and the second supply chambers 21, 22 in each of the microfluidic systems 2 via the inlet ports 32 on the top sealing layer 3, the driving device 4 drives the disk 1 to spin and thereby produces a centrifugal force, so that the first and the second fluids W1, W2 in the first and the second supply chambers 21, 22, respectively, are centrifugally flow to the first and the second receiving chambers 23, 24, at where two fluid mixtures each having a specific concentration are generated.

The first and the second fluids W1, W2 supplied into the first and the second supply chambers 21, 22, respectively, are centrifugally driven to flow through the first and the second microchannels 21a, 22a into the first mixing channels 231, as well as through the third and the fourth microchannels 21b, 22b into the second mixing channels 241. Through the Coriolis effect, the fluids W1, W2 are well mixed in both the first and the second mixing channels 231, 241.

Preferably, on each of the first, the second, the third, and the fourth microchannels 21a, 22a, 21b, 22b, there is provided at least one valve 26 located at a predetermined position near the first and the second supply chambers 21, 22. The valve 26 includes a buffering zone having a size larger than that of a corresponding microchannel 21a, 22a, 21b, or 22b. When the fluids W1, W2 are respectively supplied into the first and the second supply chambers 21, 22 and flow to the valves 26, the fluids W1, W2 are buffered without rushing into the first and the second receiving chambers 23, 24. That is, the flowing of the fluids W1 and W2 are timely limited by the valves 26.

Please refer to FIGS. 5 and 6 that are cross sectional views taken along lines 5-5 and 6-6 of FIG. 2, respectively. The first and the second microchannels 21a, 22a in each microfluidic system 2 may have different geometrical dimensions. When the first and the second fluid W1, W2 are respectively supplied into the first and the second supply chambers 21, 22, and the driving device 4 drives the disk 1 to spin and produces a centrifugal force, the first and the second fluids W1, W2 are centrifugally moved through the differently sized first and second microchannels 21a, 22a into the first receiving chamber 23 at different speeds and accordingly, thereby generate a fluid mixture having a specific concentration ratio.

When the first microchannel 21a has a geometrical size larger than that of the second microchannel 22a, the quantity of the first fluid W1 supplied from the first supply chamber 21 through the larger first microchannel 21a into the first receiving chamber 23 at a relatively quick speed is larger than the quantity of the second fluid W2 supplied from the second supply chamber 22 through the smaller second microchannel 22a into the first receiving chamber 23 at a relatively slow speed. Therefore, the concentration of the first fluid W1 in the fluid mixture in the first receiving chamber 23 is higher than the concentration of the second fluid W2 in the first receiving chamber 23. By changing the geometrical sizes of the first and/or the second microchannels 21, 22, a fluid mixture with different concentration ratio of the two fluids W1, W2 may be generated.

The second receiving chamber 24 is located between the first microchannel 21a and the second microchannel 22a, and between the first receiving chamber 23 and the first and second supply chambers 21, 22. The third and the fourth microchannels 21b, 22b may have geometrical sizes different from each other and from those of the first and the second microchannels 21a, 22a. Therefore, when the driving device 4 starts to spin the disk 1 and produce a centrifugal force, the first and the second fluids W1, W2 are driven centrifugally to flow through the third and the fourth microchannels 21b, 22b into the second receiving chamber 24 to generate a fluid mixture having a specific concentration different from that of the fluid mixture generated in the first receiving chamber 23. Again, through the Coriolis effect, the first and the second fluids W1, W2 are well mixed in both the first and the second mixing channels 231, 241.

The first and the second receiving chambers 23, 24, the first and the second microchannels 21a, 22a, and the third and the fourth microchannels 21b, 22b are arranged at different positions on the microchannel forming surface 12 of the disk 1. And, the microchannels may be otherwise designed as corrugated microchannels in order to regulate the moving speed and stay time of the fluids W1, W2 in the microchannels.

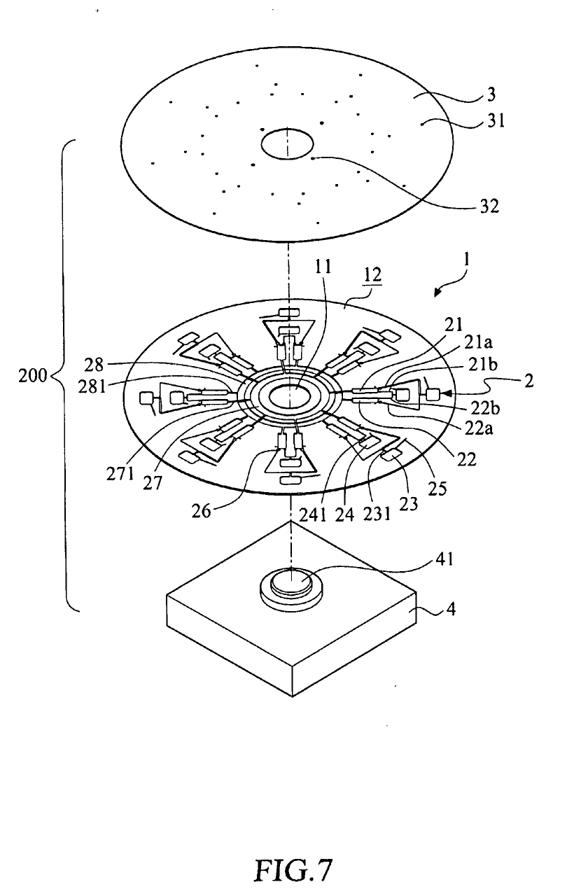

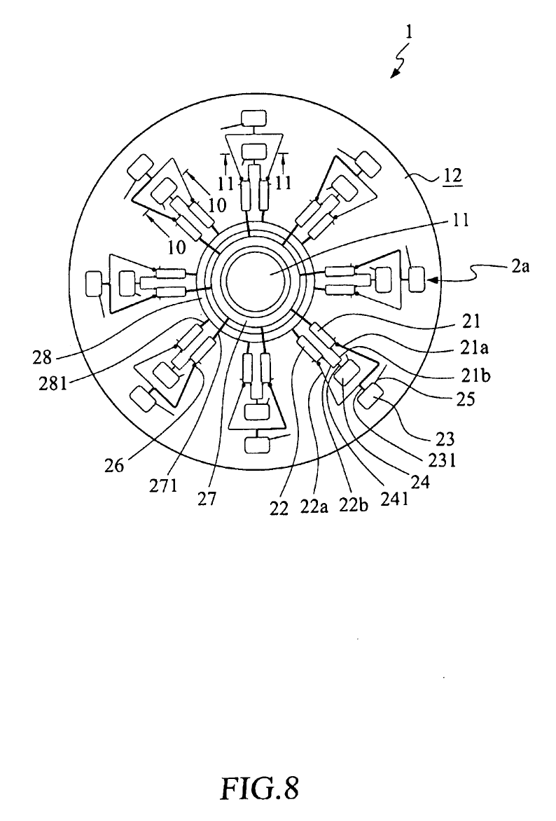

Please refer to FIG. 7 that is an exploded perspective view of a disk-like microfluidic structure 200 for generating different concentration fluid mixtures according to a second embodiment of the present invention, and to FIG. 8 that is a top view of a disk 1 included in the second embodiment of the present invention. The second embodiment is generally structurally similar to the first embodiment. Elements that are the same or similar in the first and the second embodiment are denoted with the same reference numerals. The second embodiment is different from the first embodiment in that, in addition to a plurality of microfluidic systems 2a, an inner and an outer annular supply chamber 27, 28 are formed on the disk 1 around the central opening 11 to reduce the number of times for dispensing different fluids into the plurality of first and second supply chambers 21, 22; and that the inner and the outer annular supply chamber 27, 28 are respectively communicably connected to the first and the second supply chambers 22 in each of the microfluidic systems 2a via a branch channel 271, 281, respectively.

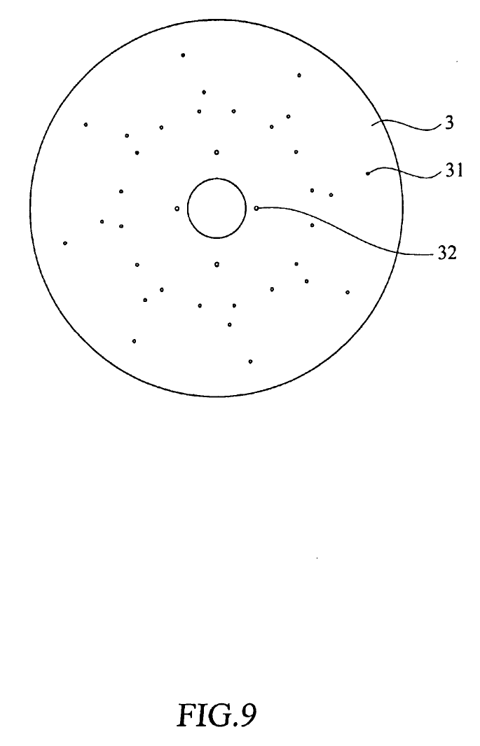

FIG. 9 is a top view of a top sealing layer 3 included in the second embodiment of the present invention. The top sealing layer 3 includes a plurality of inlet ports 32 corresponding to the inner and the outer annular supply chamber 27, 28. The first and the second fluids W1, W2 may be respectively supplied into the inner and the outer annular supply chambers 27, 28 via the inlet ports 32. When the driving device 4 starts to spin the disk 1 and produces a centrifugal force, the first fluid W1 and the second fluid W2 are centrifugally moved from the inner and the outer annular supply chambers 27, 28 into the first and the second supply chambers 21, 22 via the branch channels 271, 281, respectively. A part of the first and the second fluids W1, W2 in the first and the second supply chambers 21, 22 further centrifugally flow moved through the first and the second microchannels 21a, 22a to converge and mix at the first mixing channel 231 before being moved into the first receiving chamber 23 to generate a fluid mixture of a specific concentration, while other part of the first and the second fluid W1, W2 in the first and the second supply chambers 21, 22 further centrifugally flow through the third and the fourth microchannels 21b, 22b to converge and mix at the second mixing channel 241 before being moved into the second receiving chamber 24 to generate a fluid mixture having another specific concentration.

In the second embodiment, the microchannels 21a, 22a may be straight or bent, and may be changed in their geometrical shapes, including the length, the width, and the depth thereof, and the supply chambers 21, 22 and the receiving chambers 23, 24 may also have different geometrical shapes and locations on the disk 1, so that different concentration fluid specimens may be generated in different receiving chambers 23, 24.

Preferably, on each of the first, the second, the third, and the fourth microchannels 21a, 22a, 21b, 22b, there is provided at least one valve 26 located at a predetermined position near the first and the second supply chamber 21, 22. The valve 26 includes a buffer zone having a size larger than that of a corresponding microchannel 21a, 22a, 21b, or 22b. When the first and the second fluids W1, W2 are respectively supplied into the first and the second supply chamber 21, 22 in each microfluidic system 2a and centrifugally moved to the valves 26, the fluids W1, W2 are buffered without rushing into the first and the second receiving chamber 23, 24. That is, the moving speeds of the fluids W1 and W2 are timely limited by the valves 26.

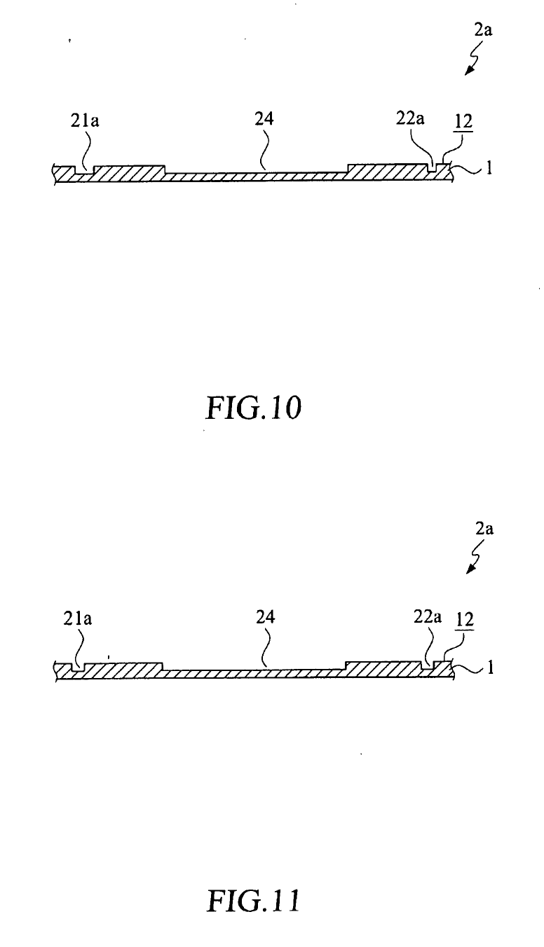

FIGS. 10 and 11 are cross sectional views taken along lines 10-10 and 11-11 of FIG. 8, respectively. When the first microchannel 21a has a geometrical size larger than that of the second microchannel 22a, as shown in FIG. 10, the concentration of the first fluid W1 in the fluid mixture generated in the first receiving chamber 23 is relatively higher than the concentration of the second fluid W2.

In the second embodiment, the second receiving chamber 24 is also located between the first and the second microchannels 21a, 22a, and between the first receiving chamber 23 and the first and second supply chambers 21, 22 to communicate with the first and the second supply chambers 21, 22 via the third and the fourth microchannels 21b, 22b. The third and the fourth microchannels 21b, 22b may have different geometrical size or the same geometrical size, which may be same as or different from that of the first and the second microchannels 21a, 22a. In the case that the microchannels 21b, 22b have the same dimension, when the driving device 4 drives the disk 1 to spin, the first and the second fluids W1, W2 centrifugally moved into the second receiving chamber 24 at the same speed. Therefore, the concentration of the first and the second fluid W1, W2 in the fluid mixture generated in the second receiving chamber 24 are the same. In other words, the geometrical sizes and structures of the first and the second microchannels 21a, 22a determines the concentration ratio of the first to the second fluid W1, W2 in the fluid mixture generated in the first receiving chamber 23. Therefore, different concentration fluid mixtures may be generated in the first and the second receiving chambers 23, 24.

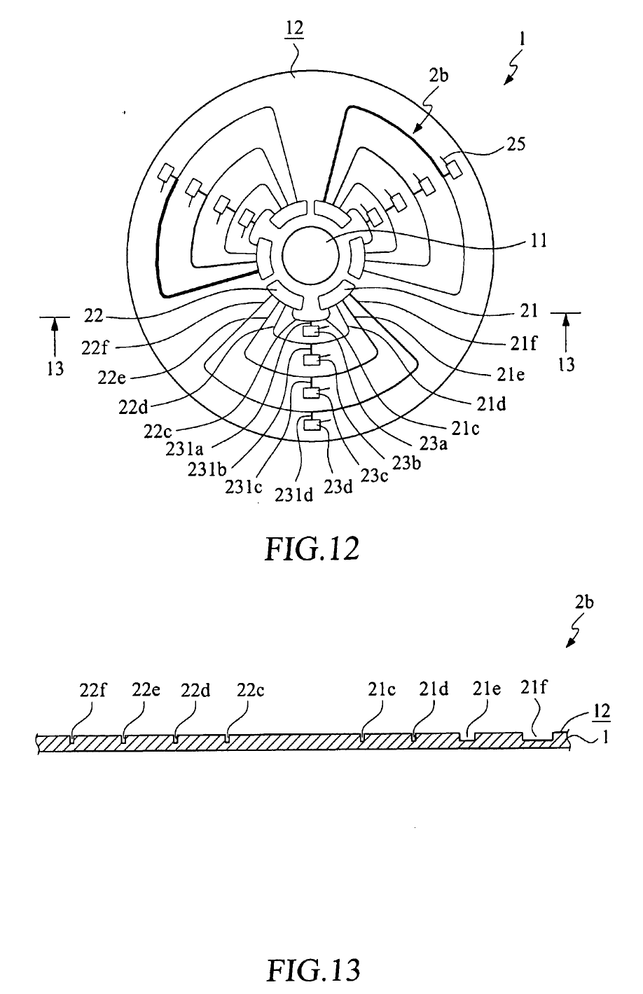

FIG. 12 is a top view of a disk 1 included in a disk-like microfluidic structure for generating different concentration fluid mixtures according to a third embodiment of the present invention; and FIG. 13 is a cross sectional view taken along line 13-13 of FIG. 12 showing differently sized microfluidic systems provided on the disk 1. The third embodiment is generally structurally similar to the first embodiment. Elements that are the same or similar in the first and the third embodiment are denoted with the same reference numerals. The third embodiment is different from the first embodiment in that the disk 1 is provided on the microchannel forming surface 12 with a plurality of microfluidic systems 2b, each of which includes a first and a second supply chamber 21, 22; a plurality of first microchannels 21c, 21d, 21e, 21f; a plurality of second microchannels 22c, 22d, 22e, 22f; a plurality of mixing channels 231a, 231b, 231c, 231d; and a plurality of receiving chambers 23a, 23b, 23c, 23d.

The plurality of first and second supply chambers 21, 22 in the microfluidic systems 2b are alternately arranged near and around the central opening 11 as a circular array. In each microfluidic system 2b, the plurality of first microchannels 21c, 21d, 21e, 21f are communicably connected to the first supply chamber 21, and the plurality of second microchannels 22c, 22d, 22e, 22f are communicably connected to the second supply chamber 22 and arranged corresponding to the first microchannels 21c, 21d, 21e, 21f.

Each first microchannel and its corresponding second microchannel (21c, 22c), (21d, 22d), (21e, 22e), (21f, 22f) are converged at a corresponding mixing channel 231a, 231b, 231c, 231d, which is then communicably connected to a corresponding receiving chamber 23a, 23b, 23c, 23d. Preferably, the plurality of receiving chambers 23a, 23b, 23c, and 23d are radially outward arranged from the central opening 11 and radially spaced by a fixed distance, so that each of the plurality of microfluidic systems 2b has a plurality of radially outward arranged and spaced microfluidic systems. Each of the plurality of receiving chambers 23a, 23b, 23c, 23d, and accordingly, the first and the second supply chamber 21, 22 communicably connected thereto are connected to a exhaust channel 25, via which gas produced in each of the microfluidic systems of the microfluidic systems 2b in the course of generating different concentration fluid mixtures is discharged.

The first microchannels 21c, 21d, 21e, 21f have geometrical sizes different from one another and from that of the second microchannels 22c, 22d, 22e, 22f. Therefore, when the driving device 4 drives the disk 1 to spin, the quantities of the first and the second fluid W1, W2 centrifugally flow from the first and the second supply chamber 21, 22 of each microfluidic systems 2b into the receiving chambers 23a, 23b, 23c, 23d vary with the geometrical sizes of the first microchannels 21c, 21d, 21e, 21f and the second microchannels 22c, 22d, 22e, 22f through which the first and the second fluid W1, W2 are centrifugally moved. That is, different concentration fluid mixtures of the first and the second fluids W1, W2 are generated in different receiving chambers 23a, 23b, 23c, 23d.

As can be easily understood by those skilled in the art, the microchannels in the microfluidic systems 2, 2a, 2b according to the disk-like microfluidic structure of the present invention may be changed in their geometrical shapes, including the length, the width, and the depth thereof; the supply chambers 21, 22 and the receiving chambers 23, 24 may also be changed in their geometrical shapes and locations on the disk 1; the microchannels in each of the microfluidic systems 2, 2a, 2b may be straight or bent microchannels; and the mixing channels 231, 241 may be straight or bent in shape, or be convergent points of a plurality of branch microchannels; so that different concentration fluid specimens may be generated at different receiving chambers 23, 24 to achieve the object of the present invention.

The microfluidic systems 2, 2a, 2b may be formed on the disk 1 in different ways, including, for example, laser sculpture, CNC (computer numerical control) machining, and microprocessing. The disk 1 may be made of a polymethyl methacrylate (PMMA) material. Alternatively, the microfluidic systems 2, 2a, 2b may be directly formed on the disk 1 by injection-molding. And, the top sealing layer may be associated with the microchannel forming surface by thermal bonding, bonding through surface modification, or adhesive bonding.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A disk-like microfluidic structure for generating different concentration fluid mixtures, comprising:
a disk, having a microfluidic forming surface; and
a plurality of microfluidic systems, which is arranged on the microfluidic forming surface of the disk as a radial array to angularly space from one another, and each of the microfluidic systems including:
a first supply chamber located near a geometrical center of the disk for holding a first fluid;
a second supply chamber located near the geometrical center of the disk for holding a second fluid, and being spaced from the first supply chamber by a predetermined distance;
a first receiving chamber located near an outer peripheral edge of the disk;
a first microchannel communicably connected at a radially inner end to the first supply chamber, and at a radially outer end to the first receiving chamber;
a second microchannel communicably connected at a radially inner end to the second supply chamber, and at a radially outer end to the first receiving chamber;
a second receiving chamber located between the first receiving chamber and the first and second supply chambers;
a third microchannel communicably connected at a radially inner end to the first supply chamber, and at a radially outer end to the second receiving chamber; and
a fourth microchannel communicably connected at a radially inner end to the second supply chamber, and at a radially outer end to the second receiving chamber;
wherein the first and the second microchannels in each of the microfluidic systems have different geometrical sizes and structures; whereby when the disk is driven by a driving device to spin, the first fluid in the first supply chamber and the second fluid in the second supply chamber are centrifugally moved into the first receiving chamber via the first and the second microchannels, respectively, and into the second receiving chamber via the third and the fourth microchannels, respectively, at different moving speeds to supply different amounts of the first and the second fluids in the first and the second receiving chambers of each microfluidic system and generate different concentration fluid mixtures.

2. The disk-like microfluidic structure for generating different concentration fluid mixtures as claimed in claim 1, wherein the first, the second, the third, and the fourth microchannel in each microfluidic system are different in length.

3. The disk-like microfluidic structure for generating different concentration fluid mixtures as claimed in claim 1, wherein the first, the second, the third, and the fourth microchannel in each microfluidic system have are different in width.

4. The disk-like microfluidic structure for generating different concentration fluid mixtures as claimed in claim 1, wherein the first, the second, the third, and the fourth microchannel in each microfluidic system have are different in depth.

5. The disk-like microfluidic structure for generating different concentration fluid mixtures as claimed in claim 1, wherein the receiving chambers and the microchannels are arranged on the microchannel forming surface of the disk at different locations.

6. The disk-like microfluidic structure for generating different concentration fluid mixtures as claimed in claim 1, wherein the microchannels are corrugated in shape arranged on the microchannel forming surface of the disk.

7. The disk-like microfluidic structure for generating different concentration fluid mixtures as claimed in claim 1, wherein the first and the second microchannels in each of the microfluidic systems are converged at a first mixing channel, which is then communicably connected to the first receiving chamber; and the third and the fourth microchannels in each of the microfluidic systems are converged at a second mixing channel, which is then communicably connected to the second receiving chamber.

8. The disk-like microfluidic structure for generating different concentration fluid mixtures as claimed in claim 1, wherein the microfluidic systems are formed on the microchannel forming surface of the disk in a manner selected from the group consisting of laser sculpture, CNC machining, microprocessing, and injection-molding.

9. The disk-like microfluidic structure for generating different concentration fluid mixtures as claimed in claim 1, wherein the microchannel forming surface of the disk is covered by a top sealing layer.

10. The disk-like microfluidic structure for generating different concentration fluid mixtures as claimed in claim 9, wherein the top sealing layer is associated with the microchannel forming surface in a manner selected from the group consisting of thermal bonding, bonding through surface modification, and adhesive bonding.

11. The disk-like microfluidic structure for generating different concentration fluid mixtures as claimed in claim 9, wherein the top sealing layer is provided with a plurality of inlet ports corresponding to the first and the second supply chambers.

12. The disk-like microfluidic structure for generating different concentration fluid mixtures as claimed in claim 9, wherein the top sealing layer is provided with a plurality of venting pores, and each of the microfluidic systems further includes a plurality of exhaust channels located corresponding to the vents on the top sealing layer to communicate with either the first or the second receiving chamber.

13. The disk-like microfluidic structure for generating different concentration fluid mixtures as claimed in claim 1, further comprising a radially inner annular supply chamber and a radially outer annular supply chamber provided on the microchannel forming surface near the geometrical center of the disk.

14. The disk-like microfluidic structure for generating different concentration fluid mixtures as claimed in claim 13, wherein the inner annular supply chamber and the outer annular supply chamber are communicably connected to the first and the second supply chambers, respectively.

15. A disk-like microfluidic structure for generating different concentration fluid mixtures, comprising:
a disk having a microchannel forming surface, and
a plurality of microfluidic systems provided on the microchannel forming surface of the disk as a radial array to angularly space from one another;
and each of the microfluidic systems including:
a first supply chamber located near a geometrical center of the disk for holding a first fluid;
a second supply chamber located near the geometrical center of the disk for holding a second fluid and being spaced from the first supply chamber by a predetermined distance;
at least one receiving chamber located closer to an outer peripheral edge of the disk;
at least one first microchannel communicably connected at a radially inner end to the first supply chamber, and at a radially outer end to the at least one receiving chamber; and
at least one second microchannel communicably connected at a radially inner end to the second supply chamber, and at a radially outer end to the at least one receiving chamber;
wherein the first and the second microchannels in each of the microfluidic systems have different geometrical sizes and structures; whereby when the disk is driven by a driving device to spin, the first fluid in the first supply chamber and the second fluid in the second supply chamber are centrifugally moved into the at least one receiving chamber via the first and the second microchannels, respectively, at different moving speeds to supply different amounts of the first and the second fluids in the at least one receiving chamber of each microfluidic system and generate a fluid mixture having a specific concentration.

16. The disk-like microfluidic structure for generating different concentration fluid mixtures as claimed in claim 15, wherein the first and the second microchannel in each of the microfluidic systems are different in length.

17. The disk-like microfluidic structure for generating different concentration fluid mixtures as claimed in claim 15, wherein the first and the second microchannel in each of the microfluidic systems are different in width.

18. The disk-like microfluidic structure for generating different concentration fluid mixtures as claimed in claim 15, wherein the first and the second microchannel in each of the microfluidic systems are different in depth.

19. The disk-like microfluidic structure for generating different concentration fluid mixtures as claimed in claim 15, wherein the at least one receiving chamber and the first and second microchannels are arranged on the microchannel forming surface of the disk at different locations.

20. The disk-like microfluidic structure for generating different concentration fluid mixtures as claimed in claim 15, wherein the first and second microchannels are waved microchannels arranged on the microchannel forming surface of the disk.

21. The disk-like microfluidic structure for generating different concentration fluid mixtures as claimed in claim 15, wherein the first and the second microchannel in each of the microfluidic systems are converged at a first mixing channel, which is then communicable connected to the at least one receiving chamber.

22. The disk-like microfluidic structure for generating different concentration fluid mixtures as claimed in claim 15, wherein the microfluidic systems are formed on the microchannel forming surface of the disk in a manner selected from the group consisting of laser sculpture, CNC machining, microprocessing, and injection-molding.

23. The disk-like microfluidic structure for generating different concentration fluid mixtures as claimed in claim 15, wherein the microchannel forming surface of the disk is covered by a top sealing layer.

24. The disk-like microfluidic structure for generating different concentration fluid mixtures as claimed in claim 23, wherein the top sealing layer is associated with the microchannel forming surface in a manner selected from the group consisting of thermal bonding, bonding through surface modification, and adhesive bonding.

25. The disk-like microfluidic structure for generating different concentration fluid mixtures as claimed in claim 23, wherein the top sealing layer is provided with a plurality of inlet ports corresponding to the first and the second supply chambers.

26. The disk-like microfluidic structure for generating different concentration fluid mixtures as claimed in claim 23, wherein the top sealing layer is provided with a plurality of venting pores, and each of the microfluidic systems further includes a plurality of exhaust channels located corresponding to the vents on the top sealing layer to communicate with the at least one receiving chamber.

27. The disk-like microfluidic structure for generating different concentration fluid mixtures as claimed in claim 15, further comprising a radially inner annular supply chamber and a radially outer annular supply chamber provided on the microchannel forming surface near the geometrical center of the disk.

28. The disk-like microfluidic structure for generating different concentration fluid mixtures as claimed in claim 27, wherein the inner annular supply chamber and the outer annular supply chamber are communicably connected to the first and the second supply chambers, respectively.

29. The disk-like microfluidic structure for generating different concentration fluid mixtures as claimed in claim 15, wherein each of the microfluidic systems on the disk includes a plurality of first microchannels, a plurality of second microchannels corresponding to the first microchannels, and a plurality of receiving chambers; and each of the first microchannels and a corresponding second microchannel thereof are communicably connected to a corresponding one of the plurality of receiving chambers.
